(19) United States
(12) Patent Application Publication
(10) Pub. No.: US 2015/0139968 A1
(43) Pub. Date: May 21, 2015

(54) PROBIOTIC COMPOSITION FOR TREATING PICORNAVIRUS INFECTION AND ITS USE THEREOF

(71) Applicant: GENMONT BIOTECH INC., TAINAN COUNTY (TW)

(72) Inventors: Ya-Fang Wang, Tainan City (TW); Jen-Ren Wang, Tainan City (TW); Yi-Hsing Chen, Tainan City (TW)

(73) Assignee: GENMONT BIOTECH INC., TAINAN COUNTY (TW)

(21) Appl. No.: 14/081,265

(22) Filed: Nov. 15, 2013

(51) Int. Cl. A61K 35/74; A23L 1/30; A61K 36/06

(52) U.S. Cl. CPC A61K 35/747; A61K 35/745; A61K 35/741; A61K 36/06; A23L 1/3014
USPC 424/93.44; 435/252.9; 424/93.45

(57) ABSTRACT

The present invention relates to a composition used for treating picornavirus infection comprising at least one of the following bacterial strains: Lactobacillus paracasei GMNL-33 with the deposition numbers of CCTCC M 206133, Lactobacillus reuteri GMNL-89 with the deposition numbers of CCTCC M 207154 and Lactobacillus casei GMNL-277 with the deposition numbers of CCTCC M 2013197, and a pharmaceutically acceptable vehicle. The Lactobacillus casei GMNL-277 of the invention is a novel Lactobacillus isolated strain. In addition, the present invention also features the novel use of the composition of the Lactobacillus strains for treating picornavirus infection, and the mechanism of which is inhibition of virus infection by binding of the probiotic bacteria to viruses.

### BACKGROUND OF THE INVENTION

1. Field of the Invention

The present invention relates to the technical fields of isolation of a novel Lactobacillus strain and its use for treating picornavirus infection.

2. Description of the Prior Art

Enterovirus (EV)-71 belongs to the Enterovirus genus of the Picornaviridae family. The viral capsid protein of EV-71 is an icosahedral structure without the envelope and viral activity is not easily affected by external pH and temperature changes; therefore, the virus can survive in the environment for a long time and infect the hosts through hand, foot and mouth transmission. The viral genome of EV-71 is a single-stranded, positive-sense RNA consisting of 7,440 bp, which is then translated into a 2,194-amino acid polyprotein containing the structural protein (P1 region) and non-structural proteins (P2 and P3 regions). This translated polyprotein is first processed by 2A protease into P1, P2, and P3, and then the structural proteins VP1, VP2, VP3 and VP4, 2A protease, 2B and 2C nucleotide triphosphatase, and the non-structural proteins 3A and 3CD are then produced by 3D protease from P1, P2 and P3, respectively. The viral capsid protein comprises 4 structural proteins VP1 to VP4 with a diameter around 20-30 nm.

Enteroviruses were re-classified into 5 groups according to their biological features as well as molecular features in 2003, including polioviruses (PVs), human enterovirus A (HEV-A) (CAV-2 to CAV-8, CAV-10, CAV-12, CAV-14, CAV-16 and EV-71), human enterovirus B (HEV-B) (CAV-9, CBV-1 to CBV-6, E-1 to E-7, E-9, E-11 to E-21, E-24 to E-27, E-29 to E-33, EV-69 and EV-73), human enterovirus C (HEV-C) (CAV-1, CAV-11, CAV-13, CAV-15, CAV-17 to CAV-22, and CAV-24), and human enterovirus D (HEV-D) (EV-68 and EV-70). EV-71 was first isolated from a patient with central nervous disease in California, United States in 1969 and was named Enterovirus (EV)-71 by Melnick in 1974.

Ever since its isolation in 1969, EV-71 has caused widespread pandemic infections across the world and resulted in severe encephalitis as well as polio-like syndrome in numerous countries such as the U.S. from 1969 to 1973, Japan in 1978, Australia (Victoria town) and Brazil in 1986, as well as Hongkong in 1986. Moreover, in 1998, the pandemic infection of EV-71 in Taiwan affected a total of 130,000 people and 405 children with severe infection were admitted to hospitals due to Hand, Foot and Mouth Disease (HFMD) and associated aseptic meningitis, encephalitis, or acute delayed paralysis, which eventually resulted in 78 deaths. The cause of death of 75% of the 405 cases with severe infection was confirmed as EV-71 infection and the EV-71 viruses were isolated from 92% of the deceased patients. Since the pandemic infection in 1998, EV-71 infection among young children occurs in Taiwan during late Spring to early Summer and Summer seasons every year, which has become an important task for public health management.

EV-71 is neuro-invasive and causes serious neurological polio-like symptoms such as limb paralysis. Because this virus usually infects newborns and young children under 3-years-old which leads to severe CNS diseases, EV-71 is regarded as a neurotropic virus that cannot be ignored after the discovery of the poliovirus. In general, enteroviruses infect the hosts through fecal-oral route and then spread to other target organs via gastrointestinal tract, in particular, the nervous system. Thus far, no effective anti-viral drugs are available for treating acute EV-71 infection. Though IVIG therapy is used clinically, its efficacy is rather limited. Therefore, at present, palliative therapy focusing on relieving the symptoms is the most widely used treatment method for treating EV-71 infection. Prevention of infectious diseases using vaccines has proved to be a valuable tool by far; however, the EV-71 vaccine remains under development and it is uncertain when the vaccine will be on the market for clinical use. Moreover, whether the selected strain used for development of the vaccine can provide cross protection against other enterovirus infections requires further investigation.

In 1908, the Russian Nobel Prize Laureate, Elie Metchnikoff, discovered that Lactobacillus found in fermented milk is beneficial to human health, and Lilly and Stilweel in 1965 further defined the probiotics as any microorganisms which can help maintain the balance of intestinal flora and improve human health. Over the years, accumulated studies have indicated that probiotics can induce immune responses in intestinal tract during microbial infections. In U.S. Pat. Nos. 7,666,407 and 7,901,926, the inventor of the present invention has specified that Lactobacillus paracasei GMNL-33 can be used for inhibition of dental diseases caused by bacterial infection and Lactobacillus reuteri GMNL-89 has anti-inflammatory effects, respectively. Nonetheless, no report has been found in literature of whether probiotics can effectively treat the symptoms resulted from enterovirus infection.

### SUMMARY OF THE INVENTION

One aspect of the invention is to provide a novel isolated strain of Lactobacillus, and the deposition numbers of Lactobacillus casei GMNL-277 are BCRC 910585 and CCTCC M 2013197.

Another aspect of the invention is to provide a pharmaceutical composition for the treatment of picornavirus infection, which comprises Lactobacillus casei GMNL-277 with the deposition numbers of BCRC 910585 and CCTCC M 2013197.

According to the invention, said the pharmaceutical composition further comprises at least one of the following strains: Lactobacillus paracasei GMNL-33 with the deposition numbers of BCRC 910314 and CCTCC M 206133 as well as Lactobacillus reuteri GMNL-89 with the deposition numbers of BCRC 910340 and CCTCC M 207154.

According to another aspect of the invention, the picornavirus of the invention is enterovirus.

According to still a further aspect of the invention, the enterovirus of the invention is Enterovirus (EV)-71.

The pharmaceutical composition of the invention is a dosage form for oral administration, and the dosage form is selected from the group consisting of solutions, suspensions, emulsions, powders, tablets, pills, syrups, lozenges, troches, chewing gums, slurries and capsules.

Another aspect of the invention is to provide a food product which comprises Lactobacillus casei GMNL-277 with the deposition number of CCTCC M 2013197.

According to the invention, said food product of the invention further comprises at least one of the following stains: Lactobacillus paracasei GMNL-33 with the deposition numbers of BCRC 910314 and CCTCC M 206133 and Lactobacillus reuteri GMNL-89 with the deposition numbers of BCRC 910340 and CCTCC M 207154.

According to the invention, said food product also further comprises at least one of the probiotic strains selected from the group consisting of Lactobacillus sp., Bifidobacterium sp., Streptococcus sp. and yeasts.

In addition, wherein the food product further comprises an edible material and said edible material comprises at least one selected from the group consisting of water, fluid milk products, milk, concentrated milk, fermented milk, yogurt, sour milk, frozen yogurt, lactic acid bacteria-fermented beverages, milk powder, ice cream, cream cheese, dry cheese, soybean milk, fermented soybean milk, vegetable-fruit juices, juices, sports drinks, confectioneries, jellies, candies, infant formulas, health foods, animal feeds, Chinese herbs and dietary supplements.

In another aspect, the present invention provides a use of probiotic composition for the manufacture of a medicament for the treatment of picornavirus infection, wherein the composition comprises at least one selected from the group consisting of Lactobacillus paracasei GMNL-33 with the deposition number of CCTCC M 206133, Lactobacillus reuteri GMNL-89 with the deposition number of CCTCC M 207154 and Lactobacillus casei GMNL-277 with the deposition number of CCTCC M 2013197.

According to another aspect of the invention, the picornavirus of the invention is enterovirus.

According to still a further aspect of the invention, the enterovirus of the invention is Enterovirus (EV)-71.

The pharmaceutical composition of the invention is a dosage form for oral administration, and the dosage form is selected from the group consisting of solutions, suspensions, emulsions, powders, tablets, pills, syrups, lozenges, troches, chewing gums, slurries and capsules.

Still another aspect of the present invention is to provide a method for treating picornavirus infection, comprising of administering an effective amount of composition, wherein the composition comprises at least one selected from the group consisting of Lactobacillus paracasei GMNL-33 with the deposition number of CCTCC M 206133, Lactobacillus reuteri GMNL-89 with the deposition number of CCTCC M 207154 and Lactobacillus casei GMNL-277 with the deposition number of CCTCC M 2013197.

According to another aspect of the invention, the picornavirus of the invention is enterovirus.

According to still a further aspect of the invention, the enterovirus of the invention is Enterovirus (EV)-71.

The pharmaceutical composition of the invention is a dosage form for oral administration, and the dosage form is selected from the group consisting of solutions, suspensions, emulsions, powders, tablets, pills, syrups, lozenges, troches, chewing gums, slurries and capsules.

Still another aspect of the present invention is to provide a composition for the treatment of picornavirus infection, wherein the composition comprises at least one selected from the group consisting of Lactobacillus paracasei GMNL-33 with the deposition number of CCTCC M 206133, Lactobacillus reuteri GMNL-89 with the deposition number of CCTCC M 207154, and Lactobacillus casei GMNL-277 with the deposition number of CCTCC M 2013197.

According to the invention, the picornavirus of the invention is enterovirus.

According to still a further aspect of the invention, the enterovirus of the invention is Enterovirus (EV)-71.

The pharmaceutical composition of the invention is a dosage form for oral administration, and the dosage form is selected from the group consisting of solutions, suspensions, emulsions, powders, tablets, pills, syrups, lozenges, troches, chewing gums, slurries and capsules.

One example of the invention is enteroviruses which comprise a group of at least 60 viruses, including polioviruses, Coxsackie A and Coxsackie B viruses, echoviruses as well as enteroviruses, etc. A number of other types of viruses were also identified in recent years and had been re-classified according to their genomic sequences.

Another example of the invention is picornaviruses which belong to the Picornaviridae family. These positive-sense RNA viruses have no envelope and their capsid proteins all have an icosahedral structure. The genomic RNAs of picornaviruses are linked to a protein at the 5′ end, which serves as the primer for RNA replication. The Picornaviridae family can be divided into 9 genera which include: Enteroviruses, the representative specie is Polioviruses; Rhinoviruses, the representative specie is Human rhinovirus A; Hepatoviruses, the representative specie is Hepatitis A virus (HAV); Cardioviruses, the representative specie is Encephalomyocarditis virus; Aphthoviruses, the representative specie is Foot-and-mouth disease virus; Parechoviruses, the representative specie is Human parechovirus; Erboviruses; Kobuviruses and Teschoviruses.

These features and advantages of the present invention will be fully understood and appreciated from the following detailed description of the accompanying Drawings.

### BRIEF DESCRIPTION OF THE DRAWINGS

In the drawings:

FIG. 1 is the microscopic image of the probiotic strain GMNL-277.

FIG. 2 shows the efficacy of the probiotic strains on virus adsorption.

### DETAILED DESCRIPTION OF THE PREFERRED EMBODIMENT

Unless defined otherwise, all technical and scientific terms used herein have the meaning commonly understood by a person skilled in the art to which this invention belongs. As used herein, the following terms have the meanings ascribed to them unless specified otherwise. The present invention will now be described more specifically with reference to the following embodiments, which are provided for the purpose of demonstration rather than limitation.

The invention features a composition used for treating picornavirus infection, comprising at least one of the following stains: Lactobacillus paracasei GMNL-33 with the deposition number of CCTCC M 206133, Lactobacillus reuteri GMNL-89 with the deposition number of CCTCC M 207154 as well as Lactobacillus casei GMNL-277 with the deposition number of CCTCC M 2013197, and a pharmaceutically acceptable vehicle. The Lactobacillus casei GMNL-277 strain of the invention is a novel Lactobacillus isolate. In addition, the invention also relates to a novel use of said composition or the Lactobacillus strains for treating picornavirus infection, which decreases cell susceptibility upon viral infection through the mechanism of binding of the probiotic GMNL-277, GMNL-33 and GMNL-89 strains to the virus.

The Lactobacillus isolate of the invention also include its subcultures or mutants, which still maintain the same characteristics, genomes or uses (for enterovirus treatment) of the strain.

According to the invention, the composition comprises the following materials, but are not limited to: foods, drinks, healthy foods, additives for animal drinking water, additives for animal feeds, medical compositions for human and animal uses, food additives, and drink additives.

The term “treatment”, “under treatment” and other similar terms refer to methods that ameliorate, improve, reduce or reverse the disease or symptoms relating to the disease which is affecting the patient as well as the methods that prevent the occurrence of a disease or any disease-related symptoms.

The term “pharmaceutically acceptable” is used herein to refer to a material or composition which shall be compatible with other ingredients in the formulation and is harmless to a patient.

The term “Picornaviridae family” as used herein, is classified as the fourth class according to the Baltimore classification, and its genome varies from 7.2 kb to 9.0 kb. The 5′ ends of the picornaviral mRNAs are linked to a protein called VPg instead of CAP, and a common poly A tail is attached at the 3′ ends. Both ends of the viral genome contain the untranslated regions (UTRs).

According to the invention, the composition can be in any applicable dosage form which is prepared by combination of the abovementioned Lactobacillus isolate and a pharmaceutically acceptable vehicle via common techniques understood by a person skilled in the art to which this invention belongs. Said dosage forms include, but are not limited to, solutions, emulsions, suspensions, powders, tablets, pills, lozenges, troches, chewing gums, slurries or any other similar or applicable dosage forms.

Additionally, said pharmaceutically acceptable vehicle may include one or more of the following reagents: solvents, emulsifiers, suspending agents, decomposers, binding agents, excipients, stabilizing agents, chelating agents, diluents, gelling agents, preservatives, lubricants, surfactants and any other similar or applicable vehicles.

Moreover, one or more of the common solubility-increasing agents, buffering agents, preservatives, coloring agents, fragrances, spices and flavoring agents may be added, in adequate amount, to the abovementioned composition if needed.

In another example of the invention, the composition provided herein can be manufactured as a food product or health product by addition of an edible material. Said edible material includes, but is not limited to, water, fluid milk products, milk, concentrated milk, fermented milk, yogurt, sour milk, frozen yogurt, lactic acid bacteria-fermented beverages, milk powder, ice cream, cream cheese, dry cheese, soybean milk, fermented soybean milk, vegetable-fruit juices, juices, sports drinks, confectioneries, jellies, candies, infant formulas, health foods, animal feeds, Chinese herbs and dietary supplements.

In addition, the novel bacterial strain discovered in the invention can also be included in a composition along with other known bacterial strains.

The composition of the invention may further include at least one of the following groups of known probiotic strains: Lactobacillus sp., Streptococcus sp., Bifidobacterium sp., and yeasts.

Furthermore, the known Lactobacillus sp. include, but are not limited to, Lactobacillus lactis, Lactobacillus acidophilus, Lactobacillus helveticus, Lactobacillus bifidus, Lactobacillus casei, Lactobacillus paracasei subsp. paracasei, Lactobacillus rhamnosus, Lactobacillus gasseri, Lactobacillus reuteri and Lactobacillus fermentum or their combinations thereof.

The known Streptococcus sp. include, but are not limited to, Streptococcus lactis, Streptococcus thermophilus, Streptococcus cremoris or their combinations thereof.

The known Bifidobacterium sp. include, but are not limited to, Bifidobacterium breve, Bifidobacterium lactis, Bifidobacterium longum, Bifidobacterium bifidum or their combinations thereof.

The known yeasts include, but are not limited to, Saccharomyces cereviseae, Candida kefyr, Saccharomyces florentinus or their combinations thereof.

Moreover, the invention also provides a method for preparation or use of a composition comprising the abovementioned Lactobacillus strains for treating picornavirus infection.

In the invention, the administering routes of the composition and the method of its use for treating picornavirus infection can be adjusted according to various needs and are not specified and the preferred method is oral administration of a suitable dosage form of the composition.

The present invention will now be described more specifically with reference to the following embodiments, which are provided for the purpose of demonstration rather than limitation.

Probiotic Strains, Cells and Viruses

Cell Culture

Human rhabdomyosarcoma (RD) cells were cultured in High-glucose-Dulbecco's Modified Eagle's Medium (DMEM) containing 10% fetal bovine serum (FBS), human colorectal adenocarcinoma (HT-29) cells were cultured in Roswell Park Memorial Institute-1640 (RPMI1640) containing 10% FBS, and human colorectal adenocarcinoma (Caco-)2 cells were cultured in High-glucose DMEM containing 20% FBS and 1.0 mM sodium pyruvate. All cells were incubated at 37° C. with 5% CO₂. For subculture, culture medium was discarded and the cells were washed with sterile PBS followed by incubation with trypsin-EDTA at 37° C. for several minutes to disperse the cells. Following the treatment, the cells were diluted with fresh cell culture medium in adequate ratios and returned to incubator for continuous culture.

Viral Culture

EV71-N4643/TW98 was used in the present study. RD cells were cultured in a 75 cm² flask until confluent before inoculation of the viruses, and the viral culture was collected when the observed cytopathic effect (CPE) is around 90% and the infected cells were then scraped off. Following freeze-and-thaw treatment and centrifugation, the supernatant was collected and stored at −80° C. in aliquots for future experiments.

Probiotic Strains

The powders of three different probiotic strains were used in this study and separately designated as GMNL-33, GMNL-89 and GMNL-277.

Experimental Design and Methods

Cytotoxicity Assay

RD cells were inoculated onto 96-well plates and cultured overnight at 37° C. The probiotic powder was prepared with the virus culture medium at the concentrations of 10⁷ and 10⁸ CFU/mL. The cells were treated for 24 hrs. followed by addition of the WST-1 agent and the absorbance of each well was measured by an ELISA reader.

Antiviral Assay

(a) Pre-treatment of cells: RD, HT-29 and Caco-2 cells were inoculated onto 12-well plates and incubated overnight at 37° C. The cells were treated with the probiotic compositions at 37° C. for 6 hrs. At the end of incubation, the cells were washed with PBS following aspiration of the supernatant and infected with EV-71 for 24 hrs. at 37° C. The infected cells and supernatant were collected and stored at −80° C. for virus quantification.

(b) Co-incubation with the virus (competition assay): RD, HT-29 and Caco-2 cells were inoculated onto 12-well plates separately and incubated overnight at 37° C. Probiotic compositions and EV-71 were added to the cells simultaneously and incubated for 24 hrs. at 37° C. The infected cells and supernatant were collected and stored at −80° C. for virus quantification.

(c) Post-treatment of the cells: RD, HT-29 and Caco-2 cells were inoculated onto 12-well plates and incubated overnight at 37° C. EV-71 was added for 6 hrs. at 37° C. to infect the cells followed by addition of probiotic compositions for 24 hrs. at 37° C. The infected cells and supernatant were collected and stored at −80° C. for virus quantification.

Virus Quantification: Median Tissue Culture Infective Dose (TCID₅₀)

RD cells were cultured in 96-well plates overnight at 37° C. A set of serial 1:10 dilutions of the sample viruses were prepared and added sequentially to the RD cells prior to incubation at 37° C. with 5% CO₂ for 24 hrs. The culture medium was discarded after 24 hrs. and ice-cold 80% acetone was added to fix the treated cells, and anti-EV-71 monoclonal antibodies was used for virus quantification in ELISA and the absorbance was measured at 450 nm. The mean absorbance of the cell control group (contains only the cells and diluted culture medium) was calculated according to the measured results, and the mean value was multiplied by 2. A positive virus infection is determined if the absorbance is larger than the calculated value; otherwise, the result is determined as negative. Following infection determination, the Reed-Muench method was used for calculation of the TCID₅₀ of the virus.

EV71 Adsorption by the Probiotics

The viral culture of 100 TCID₅₀ was mixed with the probiotics (10⁷ CFU/mL) in an 1.5 mL eppendorf and incubated at 37° C. with 5% CO₂ for 1.5 hrs. At the end of incubation, the supernatant was collected for calculation of TCID₅₀ following centrifugation for 10 min.

Statistics

The experimental results were examined by the unpaired t test to determine whether these results are statistically significant and the results were indicated as mean±SD, and p<0.05 means the result is statistically significant.

### Example 1

The inventor (GenMont Biotech Inc.) of the present invention isolated more than 600 bacterial isolates from the gastrointestinal tract of healthy adults, which allows establishment of the culture collection of the microorganism The collection locations, collection times, collectors as well as contact information of the collectors are summarized in the Table 1.

TABLE 1
Genetic Resources Information

| | Source of the | | Collection | | |
|---|---|---|---|---|---|
| | collected | Collection | location | | Contact |
| | genetic | time | (Province | Collector | information |
| | information | (yyyy/mm/dd) | City) | (Name) | of the collector |
| GMNL-33 | Healthy adults | 2004.06.03 | Taiwan | Wang | No. 8, |
| | Gastrointestinal | | Tainan | Yin-yu | Nanke 7th |
| | tract | | City | | Road, |
| | | | | | Shanhua |
| | | | | | District, |
| | | | | | Tainan City |
| GMNL-89 | Healthy adults | 2004.06.03 | Taiwan | Wang | No. 8, |
| | Gastrointestinal | | Tainan | Yin-yu | Nanke 7th |
| | tract | | City | | Road, |
| | | | | | Shanhua |
| | | | | | District, |
| | | | | | Tainan City |
| GMNL-277 | Healthy adults | 2006.01.25 | Taiwan | Wang | No. 8, |
| | Gastrointestinal | | Tainan | Yin-yu | Nanke 7th |
| | tract | | City | | Road, |
| | | | | | Shanhua |
| | | | | | District, |
| | | | | | Tainan City |

The Lactobacillus strains which can inhibit enterovirus infection based on the analysis results were selected from the Culture Collection of the invention, and the top three selected strains were further analyzed for their virus growth inhibition effects. The deposition numbers, deposition date and names of the three Lactobacillus strains are shown in Table 2. Among which, Lactobacillus paracasei GMNL-33 and Lactobacillus reuteri GMNL-89 are previously reported strains and the original copies of documents as well as relevant information including strain characteristics, proof of deposition and viability test reports can all be found in a number of international patents. On the other hand, Lactobacillus casei GMNL-277 is a novel strain identified in the invention. The characteristics, Biolog Identification System Analysis results and 16S rDNA analysis results of this novel Lactobacillus strain are further described in the embodiments of the Example 2.

TABLE 2
Bioresource Collection and Research Center (BCRC) and China Center for Type Culture
Collection (CCTCC) depositioninformation of the Lactobacillus strains of the invention.

| | | | | Disclosed information in | |
|---|---|---|---|---|---|
| Name of the strain | | Deposition number | Deposition date | patents | |
| Lactobacillus | GMNL-33 | CCTCC M 206133 | Nov. 27, 2006 | US 7666407 | EP 1955702B |
| paracasei | | BCRC 910314 | Mar. 22, 2006 | JP 4705063B | CN 101190239B |
| Lactobacillus | GMNL-89 | CCTCC M 207154 | Nov. 19, 2007 | CN 102115721B | US 7901926 |
| reuteri | | BCRC 910340 | Nov. 14, 2006 | EP 2287286B | TW 1340021 |
| | | | | TW 1346554 | US 8298526 |
| Lactobacillus | GMNL-277 | CCTCC M 2013197 | May 9, 2013 | N/A | |
| casei | | BCRC 910585 | May 16, 2013 | | |

### Example 2

### Classification of Lactobacillus casei GMNL-277

The newly isolated strain, GMNL-277, is further classified by Food Industry Research and Development Institute in Hsinchu, Taiwan and the details of the background information are described as followed: the source of the strain is human gastrointestinal tract, the medium is MRS, the culture temperature is 37° C., and the strain is non-pathogenic.

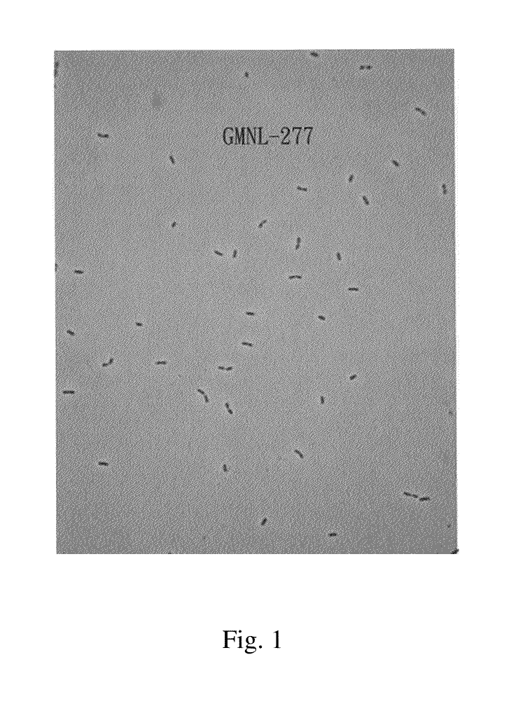

The analysis results suggested that the isolated GMNL-277 are Gram-positive bacteria which contain no catalase, no oxidase and show no motility, can grow in either aerobic or anaerobic environment and do not produce endospores (as shown in FIG. 1). In addition, part of the 16S rDNA sequence of the isolated GMNL-277 is listed in SEQ ID No: 1, and GMNL-277 and Lactobacillus casei are 99% identical in their sequences according to the 16S rDNA analysis. Further analysis using Biolog Identification System demonstrated that GMNL-277 is closely related to Lactobacillus casei. In summary, the GMNL-277 isolate is Lactobacillus casei.

### Example 3

### Inhibition of EV-71 Infection by the Probiotic Bacteria

In this study, three probiotic strains were tested in three different cell lines by three distinct treatments so as to examine the EV-71 inhibition efficiency of these probiotic strains, which include pre-treatment, co-incubation and post-treatment. Next, TCID₅₀ assay was used to assess the differences of viral load following individual treatment. From the results, the probiotics at the concentration of 10⁷ CFU/mL showed relatively no cytotoxicity effects in RD, HT-29 and Caco-2 cells, whereas concentration of 100 TCID₅₀ demonstrated the best virus inhibition effect. The results are summarized in Table 3.

TABLE 3
EV-71 Inhibition (%) of the three probiotics in three different cell lines (unpaired t test, *P < 0.05)

| | Inhibition (%) | | | | | | | | |
|---|---|---|---|---|---|---|---|---|---|
| Probiotic | Pre-treatment | | | Co-incubation | | | Post-treatment | | |
| strain | RD | HT-29 | Caco-2 | RD | HT-29 | Caco-2 | RD | HT-29 | Caco-2 |
| GMNL-277 | 65.3 ± 0.0* | 47.2 ± 20.9 | 47.1 ± 30.1 | 53.3 ± 15.0* | 38.4 ± 9.4 | 49.6 ± 20.0 | 57.9 ± 8.2 | 57.9 ± 8.2* | 44.5 ± 33.8 |
| GMNL-33 | 68.6 ± 4.6* | 55.5 ± 32.7 | 42.1 ± 22.9 | 60.1 ± 19.0* | 48.6 ± 8.4 | 53.9 ± 26.2 | 39.3 ± 34.5 | 63.7 ± 0.0* | 68.4 ± 0.0* |
| GMNL-89 | 72.9 ± 10.7* | 74.3 ± 0.0* | 58.3 ± 0.0* | 71.9 ± 3.1* | 29.2 ± 25.3 | 53.9 ± 26.2 | 57.9 ± 8.2 | 63.7 ± 0.0* | 34.2 ± 48.3 |

The results indicated that pre-treatment of the RD cells with the probiotic bacteria can inhibit EV-71 proliferation and all three strains of the invention showed statistically significant inhibition effects. In contrary, only GMNL-89 exhibited statistically significant inhibition effect when tested in HT-29 cells. On the other hand, better inhibition effect was observed in RD cells under co-incubation treatments, including GMNL-277, GMNL-33 and GMNL-89, while no notable inhibition was found in HT-29 cells under the same condition. Interestingly, the results are different in post-treatment experiments. Significant inhibition results were obtained in HT-29 cells when post-treated with GMNL-277, GMNL-33 as well as GMNL-89 while only GMNL-33 showed inhibitory effect which is statistically significant in Caco-2 cells.

Based on the abovementioned results, GMNL-277, GMNL-33 and GMNL-89 strains all have the ability to inhibit EV-71 proliferation in various cell lines. Therefore, GMNL-277, GMNL-33 as well as GMNL-89 strains were further subjected to the following virus adsorption assay as the promising key strains against enterovirus infection.

### Example 4

Examination of whether inhibition of virus replication is due to the interaction of the probiotic bacteria and EV-71:

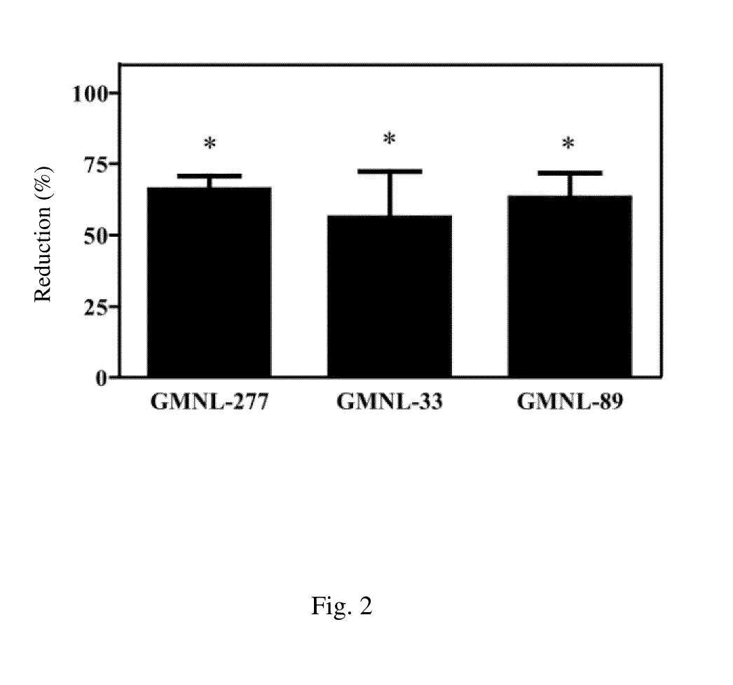

In this experiment, three promising anti-EV-71 strains were tested for their possible inhibition mechanism(s), and thus virus adsorption assay was used to assess whether the inhibition effect is due to adsorption of the virus to the probiotic bacteria. Three probiotic strains were separately co-cultured with EV-71 followed by centrifugation to precipitate the bacteria and the viral load was then quantified by using the fraction of supernatants. The results indicated that the EV-71 viral load reduced by GMNL-277, GMNL-33 and GMNL-89 was up to 66.04%, 56.09% and 63.15%, respectively (Table 4 and FIG. 2), suggesting the probiotic bacteria GMNL-277, GMNL-33 and GMNL-89 can all bind to EV-71 and therefore probiotics may inhibit viral infection by binding to viruses.

TABLE 4
Viral load reduced by adsorption of EV-71 to theprobiotic strains

| | Virus | | | |
|---|---|---|---|---|
| | control | Probiotic strain | | |
| | group | GMNL-277 | GMNL-33 | GMNL-89 |
| Plaque assay | 2.69 ± 0.08 | 2.22 ± 0.06 | 2.31 ± 0.16 | 2.25 ± 0.10 |
| (log₁₀ TCID₅₀) | | | | |
| Reduction (%) | 0 | 66.04 ± 4.7* | 56.09 ± 16.2* | 63.15 ± 8.6* |

*P < 0.05

The numerous effects demonstrated in the invention have fully satisfied the requirements of the patentability for novelty as well as non-obviousness, and therefore we request the Office to grant the patent of the invention for the purpose of invention promotion.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A novel Lactobacillus isolated strain, Lactobacillus casei GMNL-277, with the deposition number of CCTCC M 2013197.

2. A pharmaceutical composition for the treatment of picornavirus infection, which comprises Lactobacillus casei GMNL-277 with the deposition number of CCTCC M 2013197 as recited in claim 1.

3. The pharmaceutical composition used for treating picornavirus infection as recited in claim 2, wherein the composition further comprises at least one of the following strains: Lactobacillus paracasei GMNL-33 with the deposition number of CCTCC M 206133 and Lactobacillus reuteri GMNL-89 with the deposition number of CCTCC M 207154.

4. The pharmaceutical composition used for treating picornavirus infection as recited in claim 2, wherein the picornavirus is enterovirus.

5. The pharmaceutical composition used for treating picornavirus infection as recited in claim 4, wherein the enterovirus is EV-71.

6. The pharmaceutical composition used for treating picornavirus infection as recited in claim 2, wherein the pharmaceutical composition is a dosage form for oral administration.

7. The pharmaceutical composition used for treating picornavirus infection as recited in claim 6, wherein the dosage form is selected from group consisting of solutions, suspensions, emulsions, powders, tablets, pills, syrups, lozenges, troches, chewing gums, slurries and capsules.

8. A food product, which comprises Lactobacillus casei GMNL-277 with the deposition number of CCTCC M 2013197.

9. The food product as recited in claim 8, wherein the food product further comprises at least one of the following strains:
Lactobacillus paracasei GMNL-33 with the deposition number of CCTCC M 206133 and Lactobacillus reuteri GMNL-89 with the deposition number of CCTCC M 207154.

10. The food product as recited in claim 8, wherein the food product further comprises at least one of the probiotic strains selected from the group consisting of Lactobacillus sp., Bifidobacterium sp., Streptococcus sp. and yeasts.

11. The food product as recited in claim 8, wherein the food product further comprises an edible material and said edible material comprises at least one selected from the group consisting of water, fluid milk products, milk, concentrated milk, fermented milk, yogurt, sour milk, frozen yogurt, lactic acid bacteria-fermented beverages, milk powder, ice cream, cream cheese, dry cheese, soybean milk, fermented soybean milk, vegetable-fruit juices, juices, sports drinks, confectioneries, jellies, candies, infant formulas, health foods, animal feeds, Chinese herbs and dietary supplements.

12. A method for treating picornavirus infection, comprising of administering an effective amount of composition, wherein the composition comprises at least one selected from the group consisting of Lactobacillus paracasei GMNL-33 with the deposition number of CCTCC M 206133, Lactobacillus reuteri GMNL-89 with the deposition number of CCTCC M 207154 and Lactobacillus casei GMNL-277 with the deposition number of CCTCC M 2013197.

13. The method as recited in claim 12, wherein the picornavirus is enterovirus.

14. The method as recited in claim 13, wherein the enterovirus is EV-71.

15. The method as recited in claim 11, wherein the pharmaceutical composition is a dosage form for oral administration.

16. The method as recited in claim 15, wherein the dosage form is selected from the group consisting of solutions, suspensions, emulsions, powders, tablets, pills, syrups, lozenges, troches, chewing gums, slurries and capsules.


SEQUENCE LISTING (1 sequence)

SEQ ID NO: 1 - DNA, 1522 bp, Lactobacillus casei GMNL-277 strain
gatgaacgctggcggcgtgcctaatacatgcaagtcgaacgagttttggtcgatgaacggtgcttgcactgagattcgacttaaaacgagtggcggacgggtgagtaacacgtgggtaacctgcccttaagtgggggataacatttggaaacagatgctaataccgcataaatccaagaaccgcatggttcttggctgaaagatggcgcaagctatcgcttttggatggacccgcggcgtattagctagttggtgaggtaacggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcattgagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacc